(19) United States
(12) Patent Application Publication
(10) Pub. No.: US 2023/0098213 A1
(43) Pub. Date: Mar. 30, 2023

(54) DRUG-COATED BALLOON CATHETER

(71) Applicant: GENOSS CO., LTD, Suwon-si, Gyeonggi-do (KR)

(72) Inventors: Sung Min Chung, Suwon-si, Gyeonggi-do (KR); In Kwon Jung, Suwon-si, Gyeonggi-do (KR); Keun Chul Lee, Suwon-si, Gyeonggi-do (KR)

(73) Assignee: GENOSS CO., LTD., Suwon-si, Gyeonggi-do (KR)

(21) Appl. No.: 17/622,927

(22) PCT Filed: Apr. 28, 2021
(86) PCT No.: PCT/KR2021/005398

§ 371 Date: Dec. 27, 2021

(30) Foreign Application Priority Data
Jun. 3, 2020 (KR) 10-2020-0067263

(51) Int. Cl. A61M 25/10; A61L 29/16; A61M 25/09

(52) U.S. Cl. CPC A61M 25/10181; A61L 29/16; A61M 25/09; A61M 2025/105; A61L 2300/416; A61L 2300/428; A61M 2025/0081

(57) ABSTRACT

Disclosed herein is a drug-coated balloon catheter including: a hub; a soft tip; a tube which connects the hub and the soft tip and is formed of a material having higher rigidity than the soft tip; and a balloon which is mounted on the tube and coated with a drug on the surface, wherein the tube includes: a guide lumen which allows a guide wire to be movable, the guide wire being inserted into a blood vessel via the hub; and an injection lumen into which a contrast medium is injected, the contrast medium being injected into the balloon through the hub or discharged therefrom, wherein the injection lumen includes: a flow center which is a portion corresponding to a width of the guide lumen; and a pair of bend supports which is extended beyond both end portions of the guide lumen on both sides of the flow center and deformed in response to bending of the tube, and the injection lumen has a cross-sectional area more than a half of a cross-sectional area of the guide lumen.

### TECHNICAL FIELD

The present invention relates to a balloon catheter which is inserted into a blood vessel and is coated with a drug.

### BACKGROUND ART

In general, a catheter generically refers to a medical apparatus which is directly inserted into the body. In the medical field, various catheters for insertion into the body, such as catheters for vascular injection, coronary artery dilatation, urethral insertion, airway insertion, and laparoscopic surgery are used.

Among them, a balloon catheter for coronary artery dilatation has an inflatable balloon formed near a guide tube tip inserted into a blood vessel, and a fluid is introduced through a fluid infusion tube communicating with the balloon to inflate the balloon.

Meanwhile, a drug such as an anticancer drug may be applied on the surface of the balloon of the balloon catheter. The drug is configured so that when the balloon is inflated in a blood vessel and abuts against a blood vessel inner wall, the drug penetrates the blood vessel inner wall and directly acts on the blood vessel and its adjacent tissue.

In the balloon catheter as such, it is important to shorten a time taken for balloon to be inflated/deflated in terms of shortening a time taken for procedures such as vasodilation and drug application.

### DISCLOSURE OF INVENTION

### Technical Problem

An object of the present invention is to provide a drug-coated balloon catheter which greatly shortens a time used to inflate/deflate a balloon while preventing the resulting deterioration of bending resistance ability of a tube.

### Technical Solution

According to an exemplary embodiment of the present invention, there is provided a drug-coated balloon catheter including: a hub; a soft tip; a tube which connects the hub and the soft tip and is formed of a material having higher rigidity than the soft tip; and a balloon which is mounted on the tube and coated with a drug on the surface, wherein the tube includes: a guide lumen which allows a guide wire to be movable, the guide wire being inserted into a blood vessel via the hub; and an injection lumen into which a contrast medium is injected, the contrast medium being injected into the balloon through the hub or discharged therefrom, wherein the injection lumen includes: a flow center which is a portion corresponding to a width of the guide lumen; and a pair of bend supports which is extended beyond both end portions of the guide lumen on both sides of the flow center and deformed in response to bending of the tube, and the injection lumen has a cross-sectional area more than a half of a cross-sectional area of the guide lumen.

Here, along the path from one of the pair of bend supports through the flow center to the other one of the pair of bend supports, a change degree in a width of the bend support may be more than a change degree of a width of the flow center.

Here, an angle between straight lines connecting from a center point of the guide lumen to both end portions of the injection lumen may be an obtuse angle.

Here, the cross-sectional area of the injection lumen may be more than 50% and less than 60% of the cross-sectional area of the guide lumen.

Here, with respect to a cross-sectional area of the tube, a ratio of the injection lumen may be 19%, a ratio of the guide lumen may be 32%, and a ratio of the injection lumen to the guide lumen may be 59%.

### Advantageous Effects of Invention

By means of the drug-coated balloon catheter according to the present invention configured as described above, a guide lumen for insertion of a guide wire and an injection lumen to which a contrast medium for inflation/deflation of a balloon is injected are formed on the cross-section of a tube connecting a hub and a soft tip, and the injection lumen has a flow center corresponding to the guide lumen and a bend support extending from both sides of the flow center, so that the catheter has ability to resist a bending force on the tube during insertion into a blood vessel and the cross-sectional area of the injection lumen is more than a half of the cross-sectional area of the guide lumen, so that injection and recovery of the contrast medium by the injection lumen may be rapidly carried out. Thus, a time used for inflation/deflation of the balloon is greatly shortened, but the bending resistance ability of the tube may not be reduced by the bend support of the injection lumen.

### BRIEF DESCRIPTION OF DRAWINGS

FIG. 1 is a perspective view showing a drug-coated balloon catheter 100 according to an exemplary embodiment of the present invention.

FIG. 2 is a drawing for describing a balloon coating drug (D) in the drug-coated balloon catheter of FIG. 1.

FIG. 3 is a cross-sectional view of a tube 200 of a drug-coated balloon catheter 100 of FIG. 1.

FIG. 4 is a cross-sectional view of tubes 300 and 400 according to the comparative examples to be compared with the tube 200 of FIG. 3 and tested.

FIG. 5 is a graph showing test results of inflation/deflation times for the tubes illustrated in FIGS. 3 and 4.

FIG. 6 is photographs showing bending resistance ability test results for the tubes illustrated in FIGS. 3 and 4.

### MODE FOR CARRYING OUT THE INVENTION

Hereinafter, a drug-coated balloon catheter according to a preferred exemplary embodiment of the present invention will be described in detail with reference to the accompanying drawings. In the present specification, throughout the exemplary embodiments of the present invention, similar components will be denoted by the same or similar reference numerals and a description thereof will be replaced by a first description.

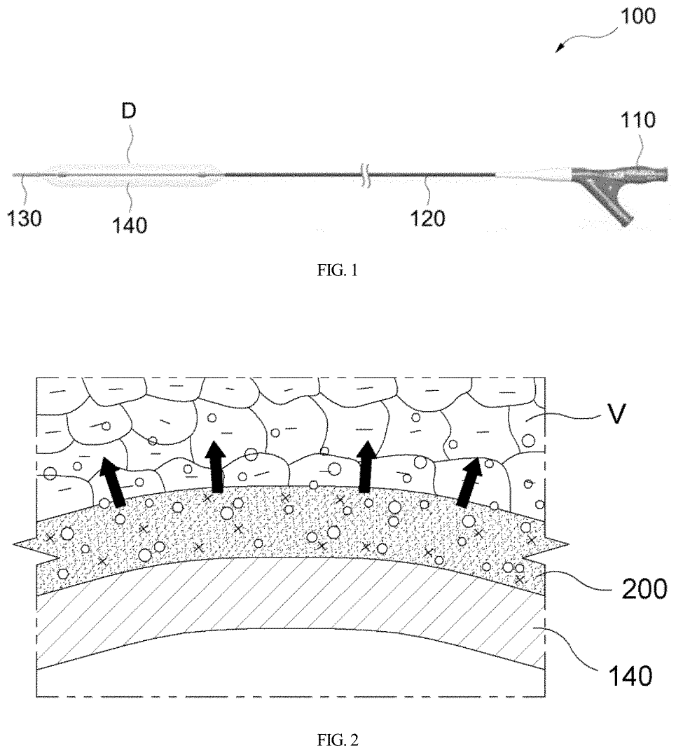

FIG. 1 is a perspective view showing a drug-coated balloon catheter 100 according to an exemplary embodiment of the present invention.

Referring to the drawing, the drug-coated balloon catheter 100 may be composed of a hub 110, a tube 120, a soft tip 130, and a balloon 140.

The hub 110 is a hollow cylindrical body. The hub 110 is formed of shape and material which an operator may hold in the hand. In terms of the material, the hub 110 may be a plastic-based material. The hub 110 may be formed of a bifurcated structure, in the present exemplary embodiment.

The tube 120 is a hollow body which is extended so as to connect the hub 110 and the soft tip 130. The tube 120 has a function to transmit a force to the soft tip 130 when an operator grabs the hub 110 and inserts the balloon 140 into a blood vessel. A portion closer to the soft tip 130 in the tube 120 is a portion which is inserted into more curved blood vessels such as a heart, and may be made of a material having high ductility so as to bend in response to the curve.

The soft tip 130 is a constituent attached to a tip of the tube 120. The soft tip 130 often collides with an inner wall of a blood vessel when entering the blood vessel from the tip of the catheter 100. In order to prevent resulting damage to the blood vessel, the soft tip 130 may be formed of a material having better ductility than the tube 120.

The balloon 140 inflates by the pressure of the contrast medium supplied through the tube 120, so that an operator may dilate the blood vessel at a desired position to improve blood vessel blockage. In addition, the operator may find out the position of the balloon 140 by the contrast medium introduced to the balloon. Here, a balloon coating drug (D) may be applied to the surface of the balloon 140.

Hereinafter, the balloon coating drug (D) will be described in detail with reference to FIG. 2.

FIG. 2 is a drawing for describing a balloon coating drug (D) in the drug-coated balloon catheter of FIG. 1.

Referring to the drawing, the balloon coating drug (D) may include an anticancer drug, an absorption enhancer, and a dissolution inhibitor.

The anticancer drug may include paclitaxel. Paclitaxel is an anticancer chemical agent extracted from a yew tree bark, and is currently widely used as an anticancer drug since approved as an anti-breast cancer drug by the US FDA in 1993. Since paclitaxel is difficult to be synthesized and is present only in a small amount in a yew tree, it is produced by obtaining by separating a baccatin III component which is present in common in various plants belonging to a yew tree family in a large amount and then subjecting the component into a semi-synthesis process. Paclitaxel functions on the M phase which is the last stage of cell division and is bound to a material called tubulin to inhibit a cell cycle, thereby showing an anti-cancer effect. The paclitaxel comes into contact with the blood vessel inner wall by the inflation of the balloon, as shown, whereby the paclitaxel may be absorbed into a vascular tissue.

An absorption enhancer is an element for promoting absorption of paclitaxel into the inner-wall of a blood vessel (V). The absorption enhancer may be composed of a vitamin E-based material. Specifically, the absorption enhancer may include vitamin E d-α-tocopheryl polyethylene glycol 1000 succinate (hereinafter, referred to as “vitamin E TPGS”). The vitamin E TPGS may have a structure of the following Chemical Formula 1:

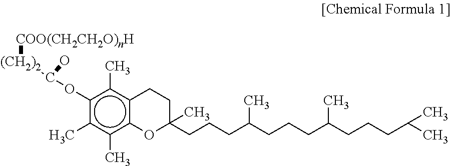

A dissolution inhibitor is an element for preventing dissolution of paclitaxel in blood. Specifically, the dissolution inhibitor may prevent paclitaxel from being dissolved in blood and the like in a blood vessel (V), while the balloon 140 in a state of being deflated in the tube 120 moves through the blood vessel (V). For this, the dissolution inhibitor may include a natural resin such as shellac. Shellac is a kind of natural resin extracted from a body fluid or secretion from a lac bug, and has excellent oil resistance and dampproof properties. Due to the properties, shellac is used in the pharmaceutical field and the like, and serves to deliver a tablet without being absorbed in the intestine, or is widely used in everyday life, for example, is used in chocolate and the like to perform anti-moisture penetration and a gloss function.

The balloon coating drug (D) composed as such may have a crystalline morphology. Specifically, in the step of preparing a balloon coating drug (D), paclitaxel described above, vitamin E TPGS, and shellac are mixed in a solvent to form a medicinal solution in a liquid state, and while the medicinal solution is stirred, a stirring speed, a temperature, and the like are adjusted according to a usual crystal production method, thereby preparing the drug so that the dried medicinal solution has a crystalline structure.

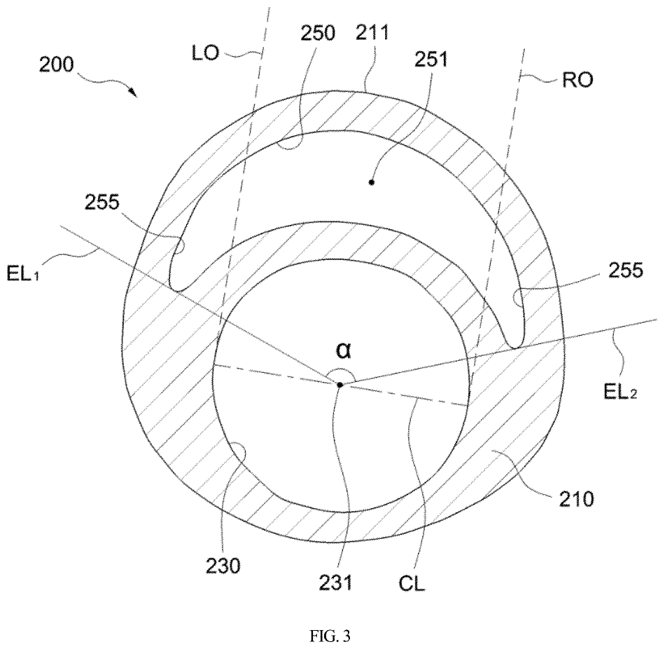

During the operation in which the drug penetrates into a blood vessel (V) and the blood vessel (V) dilates, the inflation/deflation of the balloon 140 depends on the structure of the tube 120. The tube 120 is described with reference to FIGS. 3 to 6.

First, FIG. 3 is a cross-sectional view of a tube 200 of a drug-coated balloon catheter 100 of FIG. 1. In the drawing, the reference number of the tube 120 is changed to 200 for convenience of description.

Referring to the drawing, the tube 200 may generally have a body 210 having a circular cross section. In the body 210, two lumens are formed. These lumens may be referred to as a guide lumen 230 and an injection lumen 250.

The guide lumen 230 is a lumen which houses a guide wire (not shown) inserted into a blood vessel (V, see FIG. 2) through one of two hubs 110 (see FIG. 1) to be movable. An operator first inserts the guide wire into a blood vessel through the guide lumen 230, and then pushes a catheter 100 into the blood vessel under the guidance of the guide wire. The guide lumen 230 may generally have a circular shape.

The injection lumen 250 is a lumen to which a contrast medium is injected, the contrast medium being also injected into the balloon 140 through the hub 110 (see FIG. 1) or recovered therefrom. For this, the injection lumen 250 communicates with the hub 110 and also with the balloon 140. The injection lumen 250 generally has a crescent shape, so as to cover one section of the outer periphery of the guide lumen 230.

The injection lumen 250 may be, specifically, divided into a flow center 251 and a pair of bend supports 255. The flow center 251 has a width corresponding to the width of the guide lumen 230, and the bend support 255 becomes a portion extended beyond the both end portions of the guide lumen 230. Thus, a portion positioned between a pair of straight lines (LO and RO) in contact with the outer periphery of the guide lumen 230 becomes the flow center 251 and a portion outside the pair of straight lines (LO and RO) becomes the bend support 255.

The flow center 251 has a main function to increase a flowing amount of the contrast medium by a large cross-sectional area. The pair of bend supports 255 is positioned outside the pair of straight lines (LO and RO), so that they are deformed by a portion having a small radius of curvature in the blood vessel (V) during insertion of the body 210 into the blood vessel (V). Thus, when the body 210 is bent, the bend support 255 is deformed while a farthest portion from the guide lumen 230 in the body 210 is not folded, thereby increasing resistance ability to bending by the blood vessel (V).

By the structure of the injection lumen 250, an angle (α) between straight lines (EL₁ and EL₂) extending from a center point 231 of the guide lumen 230 to both end portions of the injection lumen 250 is an obtuse angle. Furthermore, in the path from one of the pair of bend supports 255 through the flow center 251 to the other one of the pair of bend supports 255, a change degree in a width of the bend support 255 is larger than a change degree of a width of the flow center.

In addition, the cross-sectional area of the injection lumen 250 should be increased in terms of shortening a time taken for introduction and recovery of the contrast medium, but which may be problematic in terms of support for bending resistance of the body 210. The cross-sectional area of the injection lumen 250 may produce contradictory results as such, and the present inventors deduced that the lumen 250 should have a pair of bend supports 255 and have a cross-sectional area more than a half of the cross-sectional area of the guide lumen 230. Specifically, it is preferred that the cross-sectional area of the injection lumen 250 is more than 50% and less than 60% of the cross-sectional area of the guide lumen 230.

More specifically, it is most preferred that the cross-sectional area of the injection lumen 250 is 0.467 mm², considering the two performance elements described above. Furthermore, the cross-sectional area of the body 210 may be 2.425 mm² and the cross-sectional area of the guide lumen 230 may be 0.797 mm². In that case, with respect to the cross-sectional area of the body 210, a ratio of the injection lumen 250 is 19% and a ratio of the guide lumen 230 is 32%. Here, a ratio of the injection lumen 250 to the guide lumen 230 may be 59%.

A structural arrangement relationship between the injection lumen 250 and the guide lumen 230 and an advantage from the cross-sectional area ratio become clearer from other comparative examples.

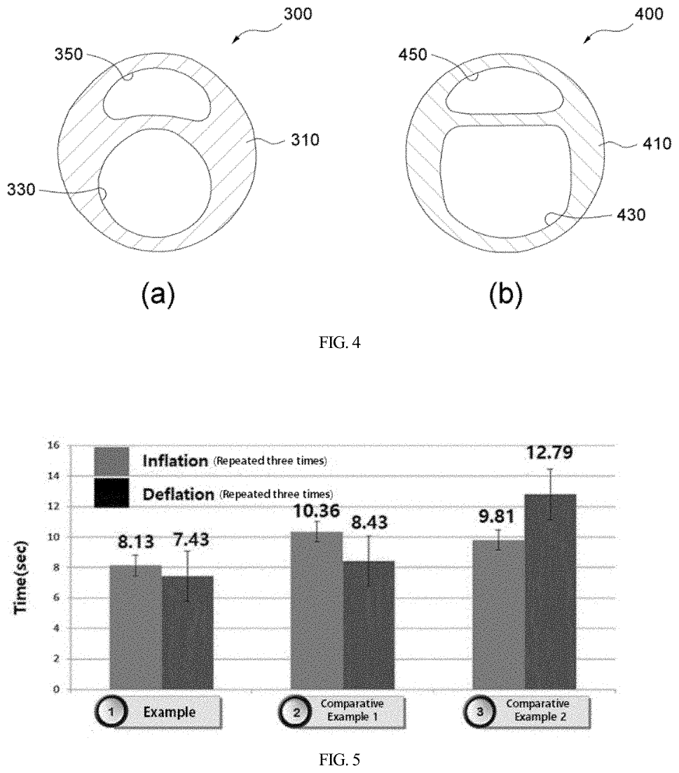

First, FIG. 4 is a cross-sectional view of tubes 300 and 400 according to the comparative examples to be compared with the tube 200 of FIG. 3 and tested.

Referring to the drawing, in the body 310 of the tube 300 of Comparative Example 1, a circular guide lumen 330 and an injection lumen 350 not out of both end portions of the guide lumen are formed. When the cross-sectional area of the tube 300 is 2.486 mm², the cross-sectional area of the former 330 is 0.779 mm² and the cross-sectional area of the latter 350 is 0.360 mm². A ratio of the latter 350 to the former 330 is 46%.

In a body 410 of a tube 400 of Comparative Example 2, a pentagonal guide lumen 430 and an injection lumen 450 which is not out of the both end portions of the guide lumen are largely formed. When the cross-sectional area of the tube 400 is 2.309 mm², the cross-sectional area of the former 430 is 0.971 mm² and the cross-sectional area of the latter 450 is 0.326 mm². A ratio of the latter 450 to the former 430 is 34%.

FIG. 5 is a graph showing test results of inflation/deflation times for the tubes illustrated in FIGS. 3 and 4.

Referring to the drawing, in the tubes 300 and 400 according to the Example and Comparative Examples 1 and 2, the balloon inflates or deflates by introduction and recovery of the contrast medium using the injection lumen 350 or 450.

In the inflation/deflation test, an inflation time was 8.13 seconds in the Example, but 10.36 seconds in Comparative Example 1 and 12.79 seconds in Comparative Example 2. In addition, a deflation time was 7.43 seconds in the Example, but 8.43 seconds in Comparative Example 1 and 12.79 seconds in Comparative Example 2.

As a result, in the inflation/deflation test, the Example had a result of about 10% to 40% reduction in the inflation/deflation time as compared with the comparative examples.

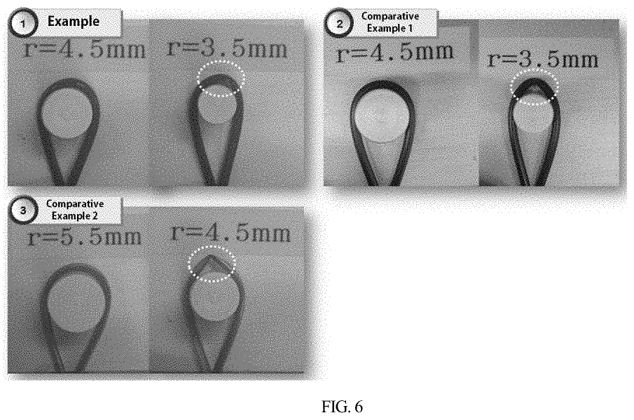

FIG. 6 is photographs showing bending resistance ability test results for the tubes illustrated in FIGS. 3 and 4.

Referring to the drawing, the Example had a little bending when a radius of curvature was 3.5 mm, but Comparative Example 1 had more pronounced bending than the Example at the same radius of curvature. In addition, it was found that Comparative Example 2 had significant bending at a radius of curvature of 4.5 mm or more.

It was found therefrom that in the Example, bending resistance was improved by the role of the bend support 255 even though the cross-sectional area of the injection lumen 250 was increased (see FIG. 3).

The drug-coated balloon catheter as described above is not limited to the configurations and the operation schemes of the above-mentioned exemplary embodiments. The above-mentioned exemplary embodiments may also be variously modified through a selective combination of all or some thereof.

### INDUSTRIAL APPLICABILITY

The present invention has industrial applicability in the balloon catheter manufacturing field.

1. A drug-coated balloon catheter comprising:
a hub;
a soft tip;
a tube which connects the hub and the soft tip and is formed of a material having higher rigidity than the soft tip; and
a balloon which is mounted on the tube and coated with a drug on the surface,
wherein the tube includes:
a guide lumen which allows a guide wire to be movable, the guide wire being inserted into a blood vessel via the hub; and
an injection lumen into which a contrast medium is injected, the contrast medium being injected into the balloon through the hub or discharged therefrom,
wherein the injection lumen includes:
a flow center which is a portion corresponding to a width of the guide lumen; and
a pair of bend supports which is extended beyond both end portions of the guide lumen on both sides of the flow center and deformed in response to bending of the tube, and
the injection lumen has a cross-sectional area more than a half of the cross-sectional area of the guide lumen.

2. The drug-coated balloon catheter of claim 1, wherein along a path from one of the pair of bend supports through the flow center to the other one of the pair of bend supports, a change degree in a width of the bend support is more than a change degree of a width of the flow center.

3. The drug-coated balloon catheter of claim 1, wherein an angle between straight lines connecting from a center point of the guide lumen to both end portions of the injection lumen is an obtuse angle.

4. The drug-coated balloon catheter of claim 1, wherein the cross-sectional area of the injection lumen is more than 50% and less than 60% of the cross-sectional area of the guide lumen.

5. The drug-coated balloon catheter of claim 1, wherein with respect to a cross-sectional area of the tube, a ratio of the injection lumen is 19%, a ratio of the guide lumen is 32%, and a ratio of the injection lumen to the guide lumen is 59%.
